(19) United States
(12) Patent Application Publication
(10) Pub. No.: US 2011/0117149 A1
(43) Pub. Date: May 19, 2011

(54) INORGANIC NANOTUBES

(75) Inventor: Jake Barralet, Montreal (CA)

(73) Assignee: Nanunanu Ltd., Longwell Green, Bristol (GB)

(21) Appl. No.: 13/002,961

(22) PCT Filed: Jul. 2, 2009
(86) PCT No.: PCT/IB2009/006720

§ 371 Date: Jan. 6, 2011

Related U.S. Application Data
(60) Provisional application No. 61/129,592, filed on Jul. 7, 2008.
(60) Provisional application No. 61/129,592, filed on Jul. 7, 2008.

(51) Int. Cl. C01B 25/26; C01B 25/32; C01B 25/42; C01B 25/40; C01B 25/44; B01J 20/24; B01J 20/04; B01J 32/00; A61K 9/00; A61K 8/02; A61K 33/42; A61K 8/24; E04B 1/74; A23J 1/00; A23L 3/00; B82Y 30/00; B82Y 40/00; B82Y 5/00

(52) U.S. Cl. 424/401; 423/305; 423/307; 423/308; 423/315; 423/314; 502/403; 502/400; 502/439; 424/400; 424/602; 252/62; 426/656; 426/321; 977/762; 977/900

(57) ABSTRACT

Discloses are stabilized calcium pyrophosphate nanotubes, a process for making calcium pyrophosphate nanotubes comprising agitating at less than 2 OkHz an aqueous suspension of a calcium and a phosphate for a time sufficient to precipitate said inorganic calcium pyrophosphate nanotubes, and uses thereof.

This application claims the benefit of U.S. Provisional Patent Application Ser. No. 61/129,592. The co-pending U.S. Provisional Application is hereby incorporated by reference herein in its entirety and is made a part hereof, including but not limited to those portions which specifically appear hereinafter.

### FIELD OF THE INVENTION

The present invention concerns inorganic nanotubes and more particularly stabilized inorganic nanotubes, a process for making the same and uses thereof.

### BACKGROUND OF THE INVENTION

Since the discovery of the carbon buckyball and the nanotube the list of both organic and inorganic materials that can be formed in one of these nanostructures is ever growing and it is now thought that almost any material can be formed into at least a one-dimensional nanotubular structure as materials that do not spontaneously form tubes can be forced into this nanostructure by templating on an existing nanotubular pore (Dachi Yang et al., “Electrochemical synthesis of metal and semimetal nanotube-nanowire heterojunctions and their electronic transport properties”, Chem. Commun., 2007, 1733-1735).

Inorganic nanotubes are attracting much interest as supports for catalysts, templates for nanowires and nanotube synthesis and nanofluidics. However, cheap large-scale production of inorganic nanotubes prevents many mass market applications from even being evaluated.

Inorganic and carbon based nanotubes are typically formed at temperatures in excess of 100° C. either by vapour deposition or hydrothermal processes, whereas organic molecules can spontaneously form nanotubes at ambient conditions. While produced easily at low temperature, such organic nanotubes cannot withstand drying and are not stable against high temperature and bacterial degradation. (Hartgerink, J. D., Granja, J. R., Milligan, R. A. & Ghadiri, M. R. Self-assembling Peptide Nanotubes, Journal of the American Chemical Society 118, 43-50, 1996).

Currently, inorganic nanotubes are grown by one or more of sulphurization of reactive oxides and halides, precursor decomposition, templated growth, pyrolysis, and direct vapor phase synthesis; and more recently via hydrothermal, sol-gel, intercalation-exfoliation, and sonochemical reactions. (Remskar M: Inorganic Nanotubes, Advanced Materials 2004, 16, 1497-1504).

Unlike organic materials that spontaneously form nanotubular structures in solution at ambient conditions, naturally occurring inorganic nanotubes that form from an inherent strain in the crystal lattice can only be prepared following a heat treatment or lengthy preparations; for example, alumino-germanate, alumino-silicate nanotubes (reflux at 95° C. for 3 h) (Sanjoy Mukherjee et al., Phenomenology of the Growth of Single-Walled Aluminosilicate and Aluminogermanate Nanotubes of Precise Dimensions, Chem. Mater., 17 (20), 4900-4909, 2005), magnesium hydroxide silicates (hydrothermal synthesis) and urynal selenides (6-7 days at room temperature) (Krivovichev, S. V. et al., Highly Porous Uranyl Selenate Nanotubes, Journal of the American Chemical Society, 127, 1072-1073, 2005).

One known inorganic nanotube, used in optics and optoelectronics, is made from TiO₂. The process for producing the TiO₂ nanotubes requires a reaction for 22 hours at a minimum temperature of 120° C. in an autoclave in 10M sodium hydroxide, or 4 h at 110° C. after sonicating for 1 hour (Y Zhu et al., Chem. Commun., 2001, 2616-2617). These relatively severe processing conditions hinder large scale manufacture.

Therefore, the progress of nanotechnology faces two limitations: (i) large scale production and, as a result, (ii) high product price. Nanotechnology is often thought of as being the preserve of high technology niche applications. However, harnessing the unique nanoscale properties, in bulk applications may be possible if an appropriate low cost process can be developed.

It is therefore desired to overcome or reduce the abovementioned problems.

### SUMMARY OF THE INVENTION

The inventor has made a significant and unexpected discovery that inorganic nanotubes, particularly calcium pyrophosphate nanotubes and polyphosphate nanotubes, can be manufactured using a process under ambient conditions. For example, rapid agitation during crystallization of an amorphous calcium pyrophosphate phase yields domains of aligned nanotubes. The new process allows the spontaneous formation of inorganic nanotubes at these ambient conditions. To our knowledge, no other inorganic nanotubes can currently be made at ambient conditions. Therefore a cost effective, environmentally-benign process for producing inorganic nanotubes, suitable for large scale manufacture, has been developed.

According to one aspect of the present invention, there is provided inorganic nanotubes.

According to another aspect of the present invention, there is provided stabilized inorganic nanotubes.

According to yet another aspect of the present invention, there is provided metal salt nanotubes.

According to still another aspect of the present invention, there is provided stabilized metal salt nanotubes.

According to one aspect of the present invention, there is provided calcium pyrophosphate nanotubes.

According to another aspect of the present invention there is provided stabilized calcium pyrophosphate nanotubes.

According to another aspect of the present invention there is provided stabilized pyrophosphate nanotubes.

According to another aspect of the present invention there is provided a composition comprising: calcium pyrophosphate nanotubes, as described above.

According to another aspect of the present invention, there is provided a composition comprising: stabilized calcium pyrophosphate nanotubes, as described above.

According to one aspect of the present invention, there is provided a process for preparing stabilized inorganic nanotubes, the process comprising: agitating an aqueous suspension of a calcium and a pyrophosphate for a time sufficient to precipitate the inorganic nanotubes. The process further includes the step of stabilizing the inorganic nanotubes. Preferably, the agitation is mechanical agitation such as shaking or bubbling. In one example, the agitation takes place at less than 800 Hz.

Typically, the step of agitation involves shaking the aqueous suspension at a frequency of less than about 20 kHz, preferably less than 100 kHz. Alternatively, the step of agitation involves bubbling gas through the aqueous suspension. Typically, the step of agitation is carried out for less than 20 minutes at room temperature. The precipitated inorganic nanotubes are selected from the group consisting of: calcium ions and pyrophosphate orthophosphate, triphosphate, polyphosphate, trimeta phosphate, hexameta phosphate, and superpolyphosphate ions. In one example, the super polyphosphate ions are from 118 polyphosphoric acid. In another example, the precipitated inorganic nanotubes contain calcium and pyrophosphate ions.

The suspension of a calcium and a phosphate can comprise calcium phosphate, calcium pyrophosphate and calcium polyphosphate.

The step of stabilization includes heat treatment and or dehydration at a temperature of typically at least 100° C., more typically at least 150° C. This is a heat based reaction, thus the lower the temperature, the longer the time. Protein coating is also possible where the nanotubes are immersed in a protein solution, for example. Also effective are polyionic anions such as 5% phytic acid; polyamino acids, for example, polyaspartic acid, amino acid, for example, aspartic acid, and a multi charged ion, for example, tripolyphosphate, (such as sodium tripolyphosphate added to the solution). The stability depends on pH. Generally speaking, phytic acid use is optimal at low pH.

According to another aspect of the present invention, there is provided calcium pyrophosphate nanotubes prepared by the process, as described above.

According to another aspect of the present invention, there is provided stabilized calcium pyrophosphate nanotubes prepared by the process, as described above.

According to yet another aspect of the present invention, there is provided use of stabilized calcium pyrophosphate nanotubes selected from the group consisting of: thermal insulating material, drug delivery, sensor, template for making nanofibres and tubes, substrate, catalyst support and absorbent, cosmetic, neutraceutital, food use, dental applications, biomaterial, implant, cosmetic surgery, wound dressing, inhalant, catalyst support, bioseparation, and a component in a composite.

With regard to the use of nanomaterials in the field of thermal insulation, silica aerogel was the first dramatic demonstration of the huge potential of the nanoscale to offer fascinating new material properties. Since its discovery, silica aerogel has only provided two large scale commercial thermal insulation products, namely light translucent beads and impregnated fabrics [1]. One of the reasons for this is high cost due to the processing needed to maintain the aerogel structure during drying, resulting in low yields.

However as we now struggle with the consequences of decades of largely unchecked consumption of energy and resources it is becoming apparent that the entire lifecycle of the materials we use, from manufacture to disposal is of great importance if we are to maintain environmental stability. Fiber glass insulation is perhaps the most widely used thermal insulation material and has many positive attributes since it delays landfill demand by providing a use for slag heaps and collected waste glass, and is vital in decreasing both domestic and industrial energy consumption. However its production creates CO₂ and waste by-products [2].

The ubiquitous nature of fiber glass and the slow market penetration of aerogel technologies highlights the need for another insulation approach but without the financial or environmental cost of both. The nanotubes of embodiments of the present invention and the method of making these nanotubes overcomes the disadvantages associated with both fiber glass and aerogel technologies.

Intuitive comparison with biological insulators tells us that “fluffy”, fibrous and air entrapping materials will be good insulators and it is pore size as well as total porosity that affects insulation. Three processes determine heat transfer in porous solids, i) solid conductivity, which increases with density ii) gaseous conductivity and iii) radiative conductivity that decreases with density. It can therefore be seen that an optimal density exists for maximum insulation. At near optimal densities at room temperature gaseous conductivity predominates. As pore size decreases from 0.1 μm to 10 nm thermal conductivity of the gas decreases by an order of magnitude. For this reason nanoporous aerogels currently have the lowest thermal conductivity in the order of 0.001 W/mK.

However interesting insulation properties have been observed in silicon nanowires; recently experimental [6] and theoretical [7] studies have shown that the thermal conductivity of Si nanowires with diameters of 22-115 nm was more than two orders of magnitude lower than their bulk value. Theoretical calculations show that this is further reduced by a factor of three by making the nanowire hollow i.e. into nanotubes [8]. Therefore, the nanotubes of embodiments of the present invention will be well suited for thermal insulation applications.

### BRIEF DESCRIPTION OF THE DRAWINGS

Further aspects and advantages of the present invention will become better understood with reference to the description in association with the following Figures, wherein:

FIGS. 1a to 1c are transmission electron micrographs of (a) high aspect ratio nanotubes, (b) lower aspect ratio nanotubes prepared by controlling reaction duration, and (c) higher magnification of the low aspect nanotubes showing an end on view of a nanotube (arrow), according to an embodiment of the present invention;

FIGS. 2a and 2b are light microscopy images of xerogels of nanotubes according to an embodiment of the present invention showing (a) crystallization following immersion of unstabilized nanotubes in water for 24 hrs (field width 300 μm), and (b) stability of nanofibrous xerogel after one month immersion in water at room temperature (field width 1 mm); and

FIGS. 3a and 3b are transmission electron micrographs of nanotubes made according to an embodiment of the invention where agitation of a starting mixture is through bubbling for a) 8, b) 12 minutes.

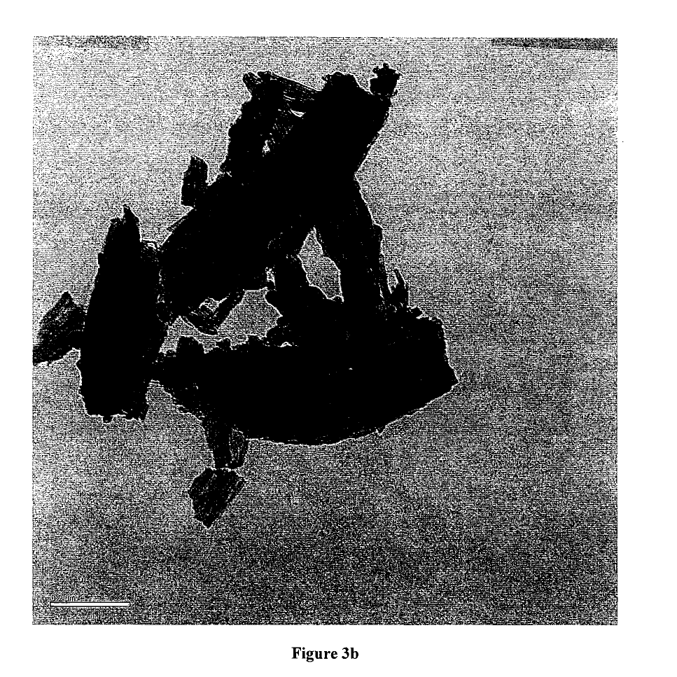

FIGS. 4a through 4e include transmission electron micrographs of preparation routes for the precipitation of amorphous calcium pyrophosphate (4a) and formation and crystallization of nanofibrous microspheres under static conditions (4b and 4c) and formation and crystallization of nanotubes with vibration (4d and 4e).

FIG. 5 include transmission electron micrographs of calcium pyrophosphate precipitates showing initial nanotube formation during vibration in an amalgamator (2 min, arrow), growth and alignment (5 min), subsequent nanotube collapse and aqueous sintering (7 and 9 min) in dense structures superficially resembling microcrystals (12 min) and final formation of microcrystalline dicalcium pyrophosphate dehydrate (15 min).

### DETAILED DESCRIPTION OF THE INVENTION

### Definitions

Unless otherwise stated, the following terms apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “nanotube” is intended to mean a hollow structure having a narrow dimension (diameter) of about 0.3 to about 160 nanometers and a long dimension (length) of about 3 nanometers or more. Generally speaking, such nanotubes have an aspect ratio of about 4:1 or more.

As used herein, the term “stabilized” is intended to mean lack of substantial microstructural and/or nanostructural changes in morphology during storage under aqueous conditions.

The terms “inorganic nanotubes” and “metal salt nanotube” are used interchangeably throughout the specification and are intended to mean nanotubes, as described above, that comprise metal salts as defined below.

As used herein the term “metal salt” is intended to mean a salt of any metal including Group 1, Group 2, transition metal, such as those in Groups 3 to 12 of the periodic table, or a Group 2 metal. The term “salt” includes, but is not limited to, halides, oxides, phosphates, pyrophosphates, polyphosphates, tungstenate, selenides, hydroxides, vanadates, sulphates, carbonates, oxylates, or organic acid anion, such as lactates, glycolates, malates and the like, or any other suitable anion.

Specific examples of metal salts of the invention include, but are not limited to, calcium pyrophosphate, calcium orthophosphate, calcium triphosphate, calcium polyphosphate, calcium trimeta phosphate, calcium hexameta phosphate and the like.

We have previously demonstrated the use of calcium pyrophosphates as a pulmonary drug delivery system in the form of microspheres of nanofibers (WO 2008/006204). The contents of WO 2008/006204 are hereby incorporated herein by reference. In the present invention, we have developed and characterized calcium pyrophosphate nanotubes. We have demonstrated experimentally that these new nanomaterials have similar properties, such as density, thermal conductivity, adsorption, porosity, and the like when compared to fiberglass. This discovery can be applied to the use of nanomaterials for a wide variety of commercial and industrial applications, such as for example, thermal insulation (for example in the building industry), filtration systems, drug delivery, template manufacture and the like.

Thus, the present invention addresses the limitations of the prior art by developing an original method of producing inorganic nanotubes and a nanomaterial family based on calcium phosphate or other suitable materials which can be cheaply and easily produced under ambient conditions. Aspects of the invention therefore reside in a process to control the assembly of these nanomaterial compounds and the resulting nanomaterials themselves. The outcome is a new family of stabilized nanomaterials that are nanotubes but are much cheaper to produce and environmentally benign than other inorganic and carbon nanotube preparations. In addition, the raw materials required to produce these new nanomaterials are abundant and cheap. The nanotubes of the present invention are made from biologically compatible, readily available and cheap materials using an environmentally benign process.

The present invention concerns the production of stabilized inorganic nanotubes rather than aggregates of nanofibres, as described in WO 2008/006204, using mechanical agitation, preferably at frequencies lower than ultrasound (approximately 20 kHz). Advantageously, the process uses relatively non-toxic reagents in an aqueous medium at room temperature and is thus inexpensive and safe. Furthermore, nanotubes have higher surface area and lower bulk density compared to nanofibres, and provide a protected environment for catalysis and such. Until now, no one has stabilized amorphous/nanocrystalline calcium pyrophosphate.

### I. PROCESS

Broadly speaking, the process for preparing the stabilized inorganic nanotubes of the present invention comprises: agitating, at less than about 20 kHz, an aqueous suspension of calcium and phosphate (e.g. pyrophosphate) compound and/or ions for a time sufficient to precipitate the inorganic nanotubes.

The calcium (or inorganic phosphate) can be selected from the group consisting of: calcium pyrophosphate, calcium orthophosphate, calcium triphosphate, calcium polyphosphate, calcium trimeta phosphate, calcium hexameta phosphate, and calcium superpolyphosphate. The super polyphosphate ions can be from 118 polyphosphoric acid. In one example, the calcium and the phosphate is calcium pyrophosphate (0.3M CaCl₂ and 0.15M Na₄P₂O₇] both adjusted to pH 7 prior to mixing. Alternatively, any other materials or reagents which goes through a gel phase or a nanoparticle phase on mixing can be used.

Generally speaking, the step of agitation involves mechanically shaking, agitating or vibrating the aqueous suspension at a frequency within the audible range of sound, i.e. less than about 20 kHz, preferably less than about 2000 Hz, more preferably less than about 1000 Hz, and typically less than about 100 Hz. In a typical example, the shaking frequency is about 60 to about 80 Hz.

In one example, the calcium and pyrophosphate solutions are placed in a sealed container and loaded into a mechanical shaker such as an amalgamator (Ultramat 2™ (SDI)) and shaken for about 30 minutes or less. The frequency of shaking is about 77 Hz and the shake amplitude is about 15 mm. In this example, the sealed container is a 1.5 ml Eppendorf filled to the top with the solutions. Alternatively, a mechanical shaker such as Dentsply Promix™ can be used, which has similar frequencies to the Ultramat™.

Manual agitation is also possible or any other mechanism to shake, agitate or vibrate the calcium and phosphate aqueous suspension for precipitation of the inorganic nanotubes. The shaking, agitation or vibration can be in two or three dimensions.

In an alternative process, the agitation involves bubbling gas through the aqueous suspension. The gas can be carbon dioxide, or any other suitable gas.

The nanotubes can be stabilized and isolated from the mixture by rapidly dehydrating the mixture, e.g. by pipetting into ethanol, filtration in ethanol, dropping onto a heated plate and the like. The step of stabilization can include heat treatment and or dehydration at a temperature of at least 50° C., typically at least 100° C. This is a heat based reaction, thus the lower the temperature, the longer the time that is required. Another stabilization method is pH adjustment. In fact, any of the stabilization methods described in WO 2008/006204 or any other known stabilization methods can be used with the nanotubes of the present invention.

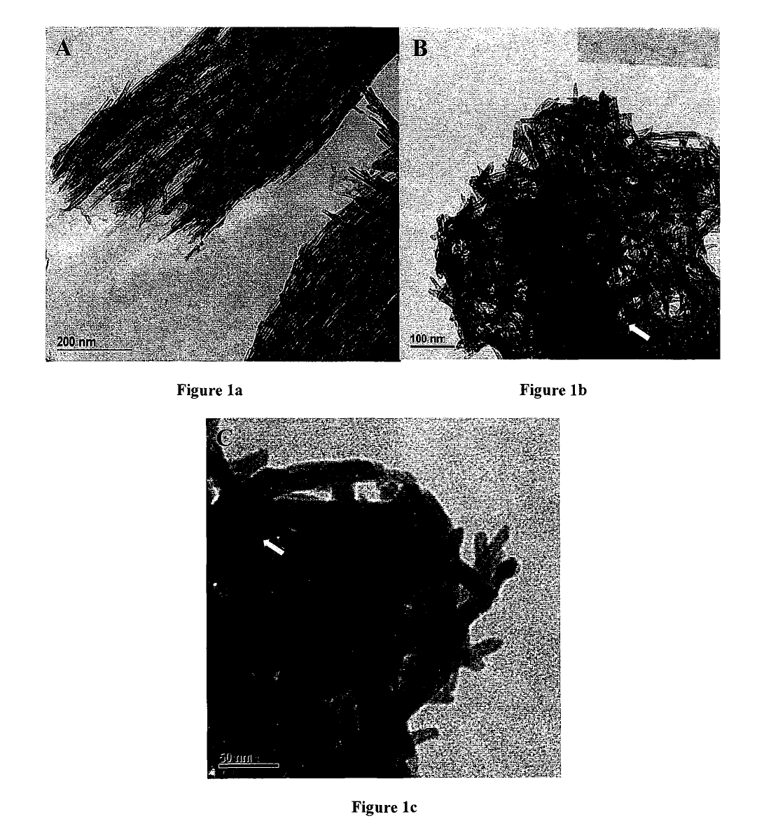

These resultant nanotubes are illustrated in FIGS. 1a to 1c. It was observed that upon addition of the reactants, a milky white precipitate is formed. A few minutes after agitation the mixture turns a transparent grey/blue colour and the mixture becomes more viscous. It has been found that mechanically agitating the aqueous suspension for less than 20 minutes, typically for several minutes, is sufficient to cause the nanotubes to precipitate. Such vibration, shaking or agitation accelerates the formation of the nanotubes from the precipitate from about 20 minutes to a few seconds.

In another example, equal volumes (15 ml) of CaCl₂, (adjusted to pH 7) and Na₄P₂O₇ (adjusted to pH 7) were added to a 50 ml measuring cylinder having a diameter of 2.1 cm. Fast bubbling was applied to the solution using a constant stream of compressed air through a tube that opened at the bottom of the cylinder for sufficient time to cause nanotube formation, for example, about 12 minutes at room temperature. Samples (less than 0.5 ml) were taken and placed directly on to a TEM grid and dried with ethanol before imaging with TEM (Transmission Electron Microscopy).

The nanotubes can be stabilized by protein coating by immersing the nanotubes in a protein solution or by any other known stabilization method, see for example WO 2008/006204. Also effective are polyionic anions such as 5% phytic acid; polyamino acids, for example, polyaspartic acid, amino acid, for example, aspartic acid, and a multi charged ion, for example, tripolyphosphate, (such sodium tripolyphosphate added to the solution). The stability depends on pH. Generally speaking, phytic acid use is optimal at low pH.

The present process has thus far provided good yields at ambient conditions. It is likely that the process is able to produce nanotubes on the kilogram scale in minutes. Therefore production of the nanotubes can be scaled-up according to demand and application. Parameters known to affect nanotube characteristics include concentration and pH of the starting reactants. Yield of the nanotubes can be maximized by maximizing the starting concentration of the reactants. To avoid batch to batch variability, a continuous reaction process can be used. Essentially water baths preheat reactants and maintain reaction temperature, flow rates, and tubing lengths determine residence and hence reaction times. Since the nanotubes are colloidal there are no issues related to particle settlement at low flow rates. Data loggers can monitor pH and temperature and manual feedback can be used to maintain constant conditions. Sampling of the mixture during the reaction may be performed to assess surface area, ion chromatography and particle size. Optical absorbance as a real time method for particle characterization can be used as an alternative. The purity of the starting reactants may affect the resultant nanotubes and this should be taken into account in order to guarantee satisfactory results.

It has also been found that shaking or agitation of any kind can induce a change in phase formed from a precipitating reaction. For example, shaking a mixture of sodium, sulphate, and calcium ions results in the precipitation of a calcium sodium sulphate, whereas in equivalent static conditions, a calcium sulphate is the product.

### II. DETAILED DESCRIPTION OF AN EMBODIMENT OF THE PROCESS

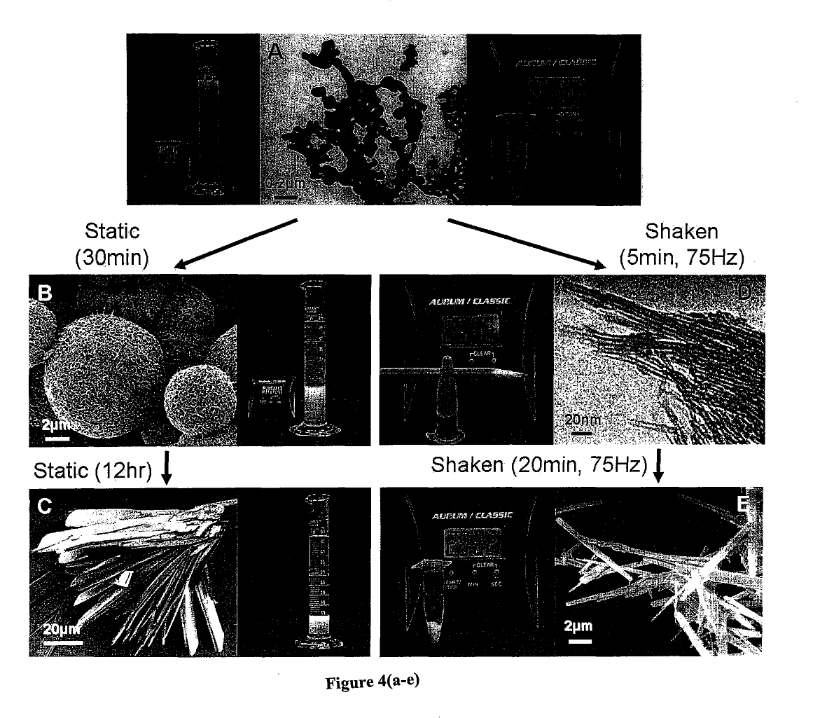

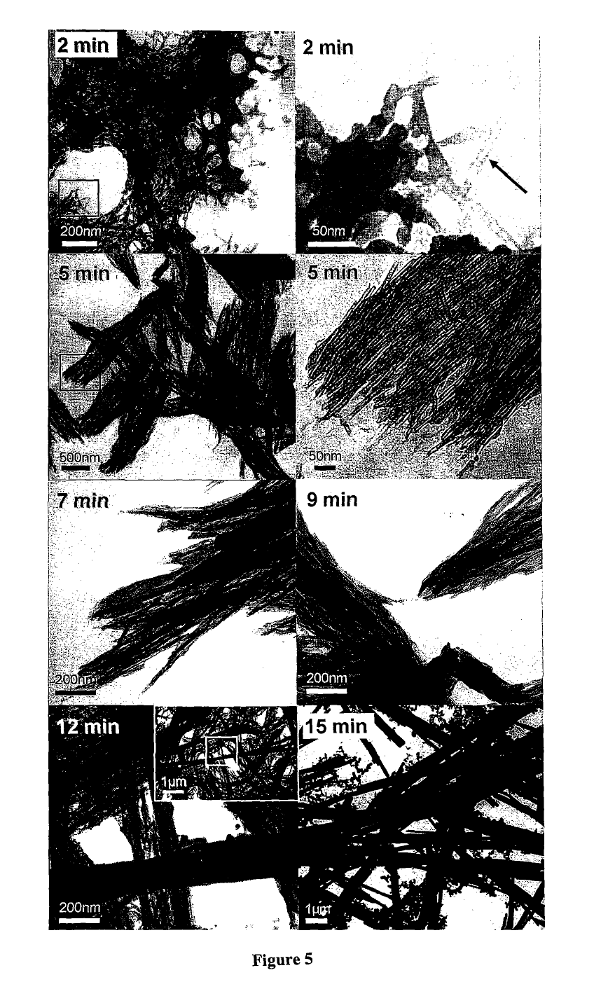

Mixing of aqueous calcium and pyrophosphate at pH 3.7 forms a milky white amorphous precipitate (FIG. 4a). Shaking the initial white amorphous precipitate resulted in the transformation to a thixotropic translucent blue-grey liquid (FIG. 4b), which then crystallised to form a mixture of tetragonal and monoclinic calcium pyrophosphate dihydrate over a period of approximately 15 min. Whereas, under static conditions, however, crystallisation took ˜2-3 hours to complete. Transmission electron microscopy (TEM) showed that the translucent liquid was a suspension of nanotubes that formed after 2 min shaking (FIGS. 4d and 5). At 5 min, microscale domains of aligned nanotubes (10 nm diameter) formed. After 7-9 min of shaking, the nanotubes collapsed and fused in a manner that we term aqueous sintering. What appeared to be microcrystals after 12 min shaking in fact consisted of fused fibres. After 15 min shaking, these assemblies densified to form microcrystals of the monoclinic and tetragonal forms of calcium pyrophosphate dihydrate some tens of microns in length (FIG. 4e). In contrast, the product of static crystallization was calcium pyrophosphate tetrahydrate. This is the first observation of crystalisation from nanotube fusion, and it is feasible that many fibrous microcrystals that form from an amorphous precursor may crystallise by this route under appropriate conditions. For comparison, FIGS. 4b and 4c show static formation of nanospheres.

Nanotubes only formed under high shear conditions (shaking and bubbling), whereas under the gentler stirring nanofibrous microspheres formed as under static, non-stirred, conditions. However, nanotube alignment was not observed under bubbling, presumably because this turbulent flow is less anisotropic than under the reciprocal shaking.

X-Ray Diffration (XRD) showed the nanotubes to be poorly crystalline/nanocrystalline. However, the nanotubes displayed greater crystallinity than the microspheres, which were formed without stirring and/or agitation. Although it has not been possible to unambiguously assign peaks within the diffraction pattern, their position closely resembles those of β-Ca₂P₂O₇. The composition of the nanotubes was determined to be Ca 26.2±0.1 wt %, P 22.7±0.1 wt %, Na 0.6±0.1 wt % giving a nominal formula Ca_{1.79}Na_{0.08}P₂O₇.2H₂O. A very weak crystallisation peak at 580° C. was observed in the nanotubular calcium pyrophosphate, indicating a more crystalline phase as confirmed by XRD. Similarly to nanofibrous microspheres, nanotubes could be stabilized by heating or storing in protein solutions.

### III. CHARACTERIZATION OF THE NANOTUBES

Transmission electron microscopy revealed a nanotubular structure (FIG. 1). Nanotubes of different lengths can be produced by varying the reaction time (see FIGS. 1a and 1b). The tubular nature of the nanotubes produced can be clearly seen in FIG. 1c. If however shaking continues eventually a microcrystalline phase settles out of solution (not shown). If the same mixture is subjected to ultrasonication instead of shaking, then microspheres are formed instead of nanotubes. This was investigated by mixing 0.5 ml of 0.3M CalCl₂ with 0.5 ml Na₄P₂O₇. The mixture was placed in a sealed container and subjected to 80 kHz ultrasound at a power of 80 W (Model G112SP1 Special Ultrasonic Cleaner) for up to 30 minutes. The formation of microparticles was delayed by approximately 10 minutes compared with the static control.

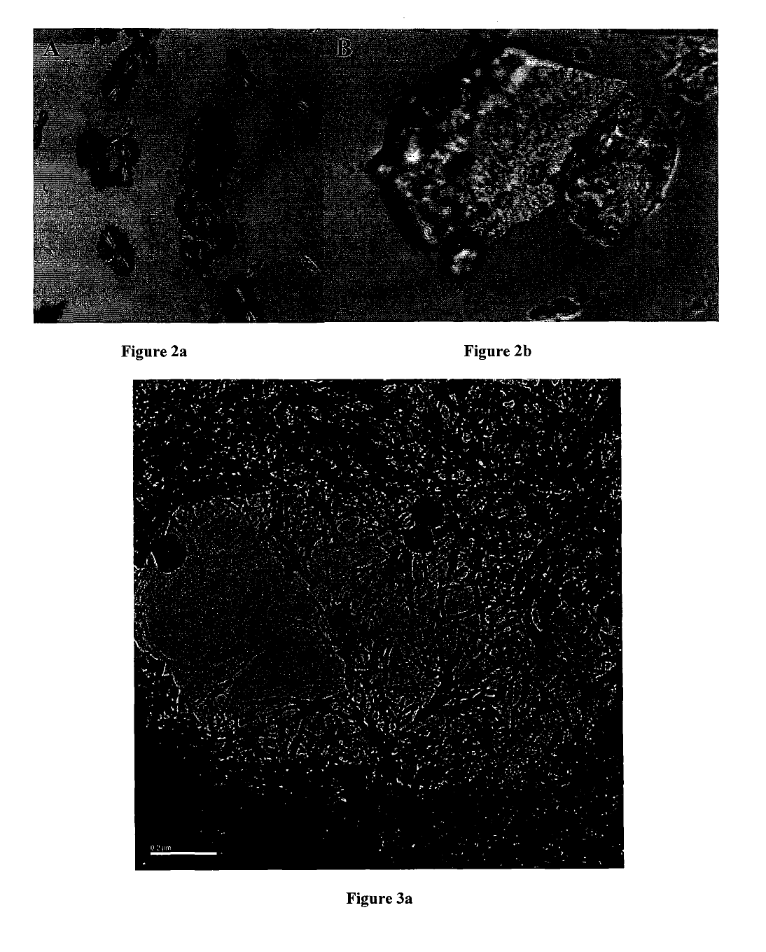

It has been confirmed that the nanotubes may be stabilized in aqueous environments for up to one month, as illustrated in FIGS. 2a and b.

The nanotubes produced according to an embodiment of the present invention have low density.

### III. UTILITIES

The nanotubes are translucent. Like many aqueous colloidal suspensions, huge shrinkage occurs on drying making the formation of cracked xerogels the most likely product of drying. We have shown that thin films can be made that do not crack on drying due to a general effect due to shear stress dependence on thickness and this offers the possibility of applying the nanotubes as translucent coatings. The use of binders enables low density blocks to be made that may be used in a similar way to polymer foams, with the added advantage of inherent non-flammability.

Previous experiments with nanofibres of a similar material (see WO 2008/006204) have shown that phagocytosis is complete within eight days of culture. i.e. the nanofibres will degrade in vivo with non-toxic degradation products. Therefore, it is expected that nanotubes of a similar material will prove to be as biodegradable and biocompatible. Therefore, the nanotubes can find application in a wide range of biomedical and biomaterial applications such as drug delivery.

The nanotubes of the present invention can be used as templates, i.e. they can be coated with other materials, semiconductors, gold etc and the nanotube template washed away to leave nanotube or desired material, or just leave the template there. For example, the stabilized calcium pyrophosphate nanotubes can be used as a template for growing other materials in fibre or tube form as is described for other tubular nanopores found in anodized alumina and carbon nanotubes and the like.

The present nanotubes are cheap to manufacture and the process to produce them is efficient and can be performed at ambient temperature. Therefore, they may be used for insulation and other bulk applications.

Moreover, the process according to embodiments of the present invention produces non-toxic by-products during synthesis.

The nanotubes can be further processed after production such as into gel granules or fibrous sheets, depending on the application. The nanotubes can form a component in a composite.

The nanotubes can be combined with a substrate or a fabric to generate nanotubes incorporated within a substrate.

Also, as the nanotubes produced by an aspect of the present invention are transparent, they can be used to coat glass, thereby insulating the glass.

Calcium pyrophosphate is readily available and inexpensive. It is bioresorbable, biodegradable and biocompatible. Calcium pyrophosphate nanotubes may be used as filtration media in filtration systems. Catalytic support and bioseparation.

### REFERENCES

[1] Fesmire J E. Aerogel insulation systems for space launch applications. Cryogenics. 2006; 46 (2-3): 111-117
[2] http://www.bau-umwelt.de/fileadmin/user_upload/Deklarationen/AUBDeklaration_DRW-10305-E.pdf
[3] W Kim, R Wang, and A Majumdar. Nanostructuring expands thermal limits, Nanotoday, 2007: 2: 40-47
[4] http://physicsworld.com/cws/article/print/23671
[5] M. Kaviany, Principles of Heat Transfer in Porous Media. Springer-Verlag, New York, 1991, p. 333
[6] Li D Y, Wu Y, Fan R, Yang P D, Majumdar A. Thermal conductivity of Si/SiGe superlattice nanowire. Applied Physics Letters. 2003; 83 (15): 3186-3188
[7] Mingo, N Calculation of Si nanowire thermal conductivity using complete phonon dispersion relations. Physical Review B. 2003; 68 (11): Art. No. 113308
[8] Huang W Q, Chen K Q, Shuai Z, Wang L L, Hu W Y. Lattice thermal conductivity in a hollow silicon nanowire. International Journal of Modern Physics B. 2005; 19 (6): 1017-1027
[9] Bonfield W (GB), Best S (GB) and Barralet J E (GB), Translucent carbonated hydroxyapatite. U.S. patent Ser. No. 08/245,679; 1993 patent granted 1995, also in Japan/Europe Applicant: QMW, University of London (GB) this patent is held in EU, US and Japan
[10] Barralet J E, Best S. Bonfield W. Carbonate Hydroxyapatite Gel Monolith formation and Drying. Biomed. Mater. and Eng., 9:101-112, 1996.
[11] Barralet J E, Aldred S. Wright A I, Coombes A G A. In vitro behaviour of albumin loaded carbonate hydroxyapatite gels. J. Biomed. Mater. Res. 60, 360-7, 2002
[12] U.S. Thermal Insulation Materials Market for the Building Industry A901-39; 2004

### Other Embodiments

While specific embodiments have been described, those skilled in the art will recognize many alterations that could be made within the spirit of the invention, which is defined solely according to the following claims:

1-28. (canceled)

29. Inorganic nanotubes comprising a phosphate.

30. The nanotubes of claim 29, wherein the phosphate is selected from the group consisting of metal phosphates.

31. The nanotubes of claim 29, wherein the phosphate comprises a pyrophosphate.

32. The nanotubes of claim 29, wherein the phosphate comprises a polyphosphate.

33. The nanotubes of claim 29, wherein the nanotubes further comprise a protein.

34. A process for preparing inorganic nanotubes, comprising the steps of:
agitating a composition comprising a liquid and a compound or ions of calcium and a phosphate for a time sufficient to cause nanotube formation; and
forming inorganic nanotubes from the composition.

35. The process of claim 34, wherein the step of agitating the composition comprises shaking, shearing or vibrating the composition at a frequency less than about 20 kHz.

36. The process of claim 34, wherein the step of agitating the composition comprises bubbling a gas through the composition.

37. The process of claim 34, wherein the compound or ions of calcium and a phosphate is selected from the group consisting of calcium phosphate, calcium pyrophosphate, calcium orthophosphate, calcium triphosphate, calcium polyphosphate, calcium triphosphate, calcium trimeta phosphate, calcium hexameta phosphate, calcium superpolyphosphate, and combinations thereof.

38. The process of claim 34, further comprising the step of stabilizing the inorganic nanotubes.

39. The process of claim 38, wherein the step of stabilizing the inorganic nanotubes comprises immersing the nanotubes in a solution.

40. The process of claim 39, wherein the solution comprises protein.

41. The process of claim 38, wherein the step of stabilizing the inorganic nanotubes comprises applying a protein to the nanotubes.

42. The process of claim 38, wherein the step of stabilizing the inorganic nanotubes comprises applying a polyelectrolyte to the nanotubes.

43. The process of claim 42, wherein the polyelectrolyte comprises a polyanion.

44. The process of claim 38, wherein the step of stabilizing the inorganic nanotubes comprises dehydrating the nanotubes.

45. The process of claim 38, wherein the step of stabilizing the inorganic nanotubes comprises heating the nanotubes.

46. A process for preparing inorganic nanotubes, comprising the steps of:
agitating a composition comprising a liquid and a compound or ions of calcium and a phosphate for a time sufficient to cause nanotube formation;
forming inorganic nanotubes from the composition; and
stabilizing the inorganic nanotubes;
wherein the inorganic nanotubes comprise a compound selected from the group consisting of calcium phosphate, calcium pyrophosphate, calcium orthophosphate, calcium triphosphate, calcium trimeta phosphate, calcium hexameta phosphate, calcium superpolyphosphate, and combinations thereof.

47. The process of claim 46, wherein the step of stabilizing the inorganic nanotubes comprises applying a protein to the nanotubes.

48. The process of claim 46, wherein the step of stabilizing the inorganic nanotubes comprises applying a polyelectrolyte to the nanotubes.

49. The process of claim 48, wherein the polyelectrolyte comprises a polyanion.
